(19) United States
(12) Patent Application Publication
(10) Pub. No.: US 2022/0265786 A1
(43) Pub. Date: Aug. 25, 2022

(54) METHODS OF IMPROVING ORGAN FUNCTION

(71) Applicant: CEDARS-SINAI MEDICAL CENTER, Los Angeles, CA (US)

(72) Inventors: Stanley Jordan, Manhattan Beach, CA (US); Jua Choi, Porter Ranch, CA (US); Ashley A. Vo, Porter Ranch, CA (US); Edmund Huang, Redondo Beach, CA (US)

(73) Assignee: CEDARS-SINAI MEDICAL CENTER, Los Angeles, CA (US)

(21) Appl. No.: 17/633,067

(22) PCT Filed: Aug. 7, 2020
(86) PCT No.: PCT/US2020/045507

§ 371 Date: Feb. 4, 2022

Related U.S. Application Data
(60) Provisional application No. 62/884,583, filed on Aug. 8, 2019.

(51) Int. Cl. A61K 38/55; A61P 37/06

(52) U.S. Cl. CPC A61K 38/55; A61P 37/06

(57) ABSTRACT

Described herein are methods for improving long-term allograft survival and long-term kidney function in subjects that have undergone kidney transplant. An effective amount of one or more complement component 1 esterase inhibitors is administered to the subject, resulting in long-term improvement on kidney allograft survival and function.

### CROSS-REFERENCE TO RELATED APPLICATIONS

This application claims priority to and benefit of U.S. provisional patent application No. 62/884,583, filed Aug. 8, 2019, the entirety of which is hereby incorporated by reference.

### FIELD OF INVENTION

The invention relates to improving organ function in subjects that have undergone organ transplant.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Delayed graft function (DGF), defined as the need for dialysis in the first week after kidney transplant, is estimated to occur in over 20% of deceased donor kidney transplants. Its development is associated with an increased risk of rejection, poorer long-term kidney allograft function, and lower patient and graft survival. This association is modified by the severity of DGF, as indicated by the duration of dialysis-dependence after transplant, where longer periods of dialysis-dependence are associated with progressively higher hazards of rejection and graft failure. Kidneys at higher risk for DGF are more likely to be discarded in the United States despite the well-documented shortage in donor organ supply.

The predominant mechanism of DGF is ischemia-reperfusion injury. This is marked by an alloantigen-independent inflammatory response, characterized by influx of pro-inflammatory cells early after ischemic injury. Additionally, the complement cascade can be activated in response to ischemia-induced membrane changes. Although the alternative pathway has historically been thought to play the major role in ischemia-reperfusion injury, evidence indicates that the classical and mannose binding lectin (MBL) pathways are also important. Damage-associated molecular patterns (DAMPs), polysaccharides, and intracellular antigens exposed during ischemic injury can activate both the classical and MBL pathways. C4-deficient mice, which cannot activate the classical pathway C3 convertase (C2aC4b), were less susceptible to ischemia-induced injury compared to wild-type mice and antibodies against mannan-binding lectin (MBL)-associated serine protease (MASP)-2 were protective against ischemia-reperfusion injury in the murine gastrointestinal tract and myocardium. Recent data from an animal model of heart transplant ischemia-reperfusion injury demonstrated that ischemia-reperfusion injury was largely prevented in animals that were genetic knockouts for the MBL collectin-11 but not for Factor B (alternative pathway) knockouts. In addition, wild-type mice treated with C1 esterase inhibitor were protected from ischemia-reperfusion injury, similar to collectin-11 (−/−).

There remains a need for the improvement of allograft function after implantation.

### SUMMARY OF THE INVENTION

The following embodiments and aspects thereof are described and illustrated in conjunction with compositions and methods which are meant to be exemplary and illustrative, not limiting in scope.

Methods are provided for improving long-term allograft survival in a subject in need thereof, which include administering to the subject a therapeutically effective amount of a C1 esterase inhibitor, wherein the subject will undergo allograft transplantation or has undergone kidney transplantation, and wherein the long-term allograft survival is in some embodiments, more than 1 year after transplantation; in some embodiments, more than 90 days after transplantation; and in some embodiments, more than 3.5 years after transplantation.

In some embodiments, the allograft is a kidney. In some embodiments, the allograft is from an expanded criteria donor.

In various embodiments, the C1 esterase inhibitor is complement C1 esterase inhibitor. Exemplary C1 esterase inhibitors include but are not limited to plasma-derived BERINERT®, plasma-derived CINRYZE®, recombinant RUCONEST® or RHUCIN®.

Various embodiments of the methods include administering the therapeutically effective amount of C1 esterase inhibitor on the day of the transplantation prior to reperfusion of the allograft, about 24 hours after transplantation, or both; which optionally is administered intravenously or subcutaneously.

In some embodiments of the methods, the therapeutically effective amount is about 25-100 units/kg, e.g., about 50 units/kg.

Some embodiments of the methods provide that the subject is at risk of developing delayed graft function (DGF), or the subject exhibits DGF or signs of graft dysfunction after allograft transplant which is effectively treated, inhibited and/or reduced after the C1 esterase inhibitor is administered to the subject and after at least 1 year, 2 years, 3.5 years or longer post-transplantation.

Further embodiments provide that the methods include administering a first dose of the C1 esterase inhibitor intraoperatively to the subject prior to reperfusion of the allograft and a second dose of the C1 esterase inhibitor about 24 hours after transplantation of the allograft, wherein the C1 esterase inhibitor is no longer administered after about three days after transplant, and wherein the allograft function is improved at least for 3.5 years after the transplant, characterized by there being no graft failure, no detectable de novo donor specific antibodies, and improved estimated glomerular filtration rates at 3.5 years after the transplant.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the study flowchart. There were a total of 70 patients randomized, 35 each to the C1 esterase inhibitor and placebo groups. No patients were excluded from the analysis.

FIG. 2A depicts cumulative incidence of graft failure and death among placebo-treated and C1 esterase inhibitor-treated patients. There was a higher incidence of graft failure and no difference in the incidence of death over 3.5 years in the placebo group compared to the C1 esterase inhibitor group. Event-free survival was not different between the groups. *Numbers at risk pertain to event-free survival. **P-values: Graft loss=0.03; Death=0.09; Event-Free=0.31

FIG. 2B depicts Kaplan-Meier curve for time to patient death; and FIG. 2C depicts cumulative incidence of graft failure comparing C1 esterase inhibitor-treated and placebo-treated patients.

FIG. 3 depicts Kaplan-Meier curve for rejection-free survival comparing C1 esterase inhibitor-treated and placebo-treated patients.

FIG. 4 depicts comparison of eGFR over time among C1 esterase inhibitor-treated and placebo-treated patients.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein the term “about” when used in connection with a referenced numeric indication means the referenced numeric indication plus or minus up to 10% of that referenced numeric indication, unless otherwise specifically provided for herein. For example, the language “about 50%” covers the range of 40% to 60%. In various embodiments, the term “about” when used in connection with a referenced numeric indication can mean the referenced numeric indication plus or minus up to 9%, 8%, 7%, 6%, 5%, 4%, 3%, 2%, 1%, 0.5%, 0.4%, 0.3%, 0.2% or 0.1% of that referenced numeric indication, if specifically provided for in the claims.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of kidney allograft function, delay or slowing of renal function, and amelioration or palliation of symptoms associated with end stage renal disease.

The terms “C1-Inhibitor,” “C1 esterase Inhibitor,” “C1-INH” and “C1INH” refer to a serine protease inhibitor that inhibits proteases associated with the complement, contact, fibrinolytic and/or coagulation systems, such as proteases C1r and C1s in the classical pathway as well as MASP-1 and MASP-2 in the MBL complement pathway, or associated with the kallikrein-kinin system, such as plasma kallikrein and factor XIIa, and or associated with the coagulation system, such as factor XIa. In addition, C1-INH can serve as an anti-inflammatory molecule that reduces the selectins-mediated leukocyte adhesion to endothelial cells. C1-INH as used herein can be a native serine protease inhibitor, as a protein or active fragment thereof, or it can comprise a recombinant peptide, a synthetic peptide, peptide mimetic, or peptide fragment that provides similar functional properties—e.g., the inhibition of proteases C1r and C1s, and/or MASP-1 and MASP-2 and/or factor XIIa and/or factor XIa. For further disclosure regarding the structure and function of C1-Inhibitor, see U.S. Pat. Nos. 4,915,945; 5,939,389; 6,248,365; 7,053,176; and WO 2007/073186, also U.S. Pat. No. 10,532,087; the content of all of which is hereby incorporated by reference.

In some embodiments, the inhibitor is a plasma-derived or a recombinant C1-Inhibitor. In a further preferred embodiment said inhibitor is identical with the naturally occurring human protein or a variant thereof. The C1-INH shall encompass all natural occurring alleles which have the same function as the C1-inhibitor. In one embodiment said inhibitor is the human C1 Esterase Inhibitor.

In another embodiment the C1-inhibitor according to the present invention is modified to improve bioavailability and/or half-life, to improve efficacy and/or to reduce potential side effects. The modification can be realized by recombinant or other steps. Examples for such a modification could be a glycosylation or an albumin fusion of the described C1-inhibitor. For further disclosure regarding the glycosylation and the albumin fusion of proteins see WO 01/79271, also U.S. Pat. No. 6,905,688, which is hereby incorporated by reference.

In various embodiments, C1-Inhibitor can be produced according to methods known to one of skill in the art. For example, plasma-derived C1-INH can be prepared by collecting blood plasma from several donors. Donors of plasma should be healthy as defined in the art. Preferably, the plasma of several (1000 or more) healthy donors is pooled and optionally further processed. An exemplary process for preparing C1-inhibitor for therapeutic purposes is disclosed in U.S. Pat. No. 4,915,945, the disclosure of which is hereby incorporated in its entirety. Alternatively, in some embodiments C1-INH can be collected and concentrated from natural tissue sources using techniques known in the art. Commercially available products comprising C1-inhibitor are, e.g. plasma-derived CINRYZE® (Viropharma), recombinant RUCONEST® or RHUCIN® (both Pharming), and plasma-derived BERINERT® (CSL Behring). BERINERT® is indicated for treatment of hereditary angioedema and congenital deficiencies. Recombinant C1-INH can be prepared by known methods.

As used herein, the term “administering,” refers to the placement of an agent or a composition as disclosed herein into a subject by a method or route which results in at least partial localization of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In an embodiment, agent or composition may be provided in a powder form and mixed with a liquid, such as water, to form a beverage. In accordance with the present invention, “administering” can be self-administering. For example, it is considered as “administering” that a subject consumes a composition as disclosed herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In an embodiment, the subject is human. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, whether male or female, are intended to be included within the scope of this term.

A subject can be one who has been previously diagnosed with or identified as suffering from or having a condition in need of treatment (e.g., kidney failure) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. For example, a subject can be one who exhibits one or more symptoms for a condition or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

In some embodiments, the subject is in need of an allograft transplantation, is a subject who has undergone a previous kidney transplant.

A therapeutically or prophylactically significant reduction in a symptom is, e.g. at least 10%, at least 20%, at least 30%, at least 40%, at least 50%, at least 60%, at least 70%, at least 80%, at least 90%, at least 100%, at least 125%, at least 150% or more in a measured parameter as compared to a control or non-treated subject or the state of the subject prior to administering C1 esterase inhibitor. Measured or measurable parameters include clinically detectable markers of disease, for example, elevated or depressed levels of a biological marker, as well as parameters related to a clinically accepted scale of symptoms or markers for fibrosis and/or inflammation. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

“Allograft survival” as used herein with respect to a kidney refers to the ability of a kidney to function without having the subject returning to the need for dialysis. “Returning to the need of dialysis” does not refer to a subject's initial need, in some cases, for dialysis immediately (e.g., 0-14 days) after transplantation. For heart and lung transplants, allograft rejection equates with death. This is particularly true if the patients progress rapidly and are not responsive to anti-rejection therapy. Liver transplants usually fail more slowly and may allow time for re-transplantation, but rapid failure is also equivalent with death. Pancreas transplant rejection equates with recurrent diabetes and need for insulin use.

“Delayed graft function (DGF)” with respect to a renal transplant, or kidney allograft, refers to a failure of a fall in serum creatinine of at least 10% on 3 consecutive days in the first post-transplant week and/or a serum creatinine level at post-transplant day 7 of greater than 2.5 mg/dL.

An “expanded criteria donor” or “extended criteria donor”, (ECD), with respect to a kidney donor generally follows the OPTN-approved criteria. In some embodiments, ECD kidney donor refers to a donor over the age of 60, or a donor over the age of 50 with two of the following: a history of high blood pressure, a creatinine (blood test that shows kidney function) greater than or equal to 1.5 mg/dL, and death resulting from a stroke. In other embodiments, ECD kidney donor refers to a donor condition of any one of the following: for a donor age ≥50, cerebrovascular accident was the cause of death (CVA)+history of hypertension (HTN)+creatinine >1/5 md/dL; for a donor age ≥50, CVA+HTN; for a donor age ≥50, CVA+creatinine >1.5 md/dL; for a donor age ≥60, CVA or HTN or creatinine >1.5 md/dL.

An “expanded criteria deceased donor” refers to an expanded criteria donor that is deceased.

The term “expanded” or “extended” is used because an expansion of the donor pool is considered to increase transplantation. Sometimes the organs are also termed ‘marginal organs’. In some embodiments, lower-quality organs or organs with greater preoperative damage are pre-treated (e.g., prior to transplantation to recipients) or preserved and/or conditioned in order to ameliorate ischemia and reperfusion injury in the process of transplantation or allow for assessment of viability and function prior to transplantation. In some embodiments, an organ is preserved or conditioned using oxygenated hypothermic or normothermic machine perfusion; and the organ may be from an expanded criteria donor. In some embodiments, an organ is modified prior to transplantation, including immunological modification; and the organ may be from an expanded criteria donor.

An expanded criteria donor can also refer to an expansion of donor pools for pancreas transplant, heart transplant, small bowel (intestinal) transplant, lung transplant, bone marrow transplant, or another solid organ transplant, or simultaneous kidney pancreas transplantation. Traditional or standard donor criteria include those guidelines provided by medical associations or organizations.

An expanded criteria donor with respect to a liver donor commonly refers to (1) a donor after brain death with: cardiac arrest >15 minutes, prolonged hypotensive periods of <60 mmHg for >1 hour, age >55 years, BMI>30 kg/m², hepatitis B virus positive, hepatitis C virus positive, macrosteatosis >30%, hypernatriemia >155 mEq/L, intensive care unit stay ≥5 days, nosocomial infection with positive blood cultures or pneumonia, split liver, aspartate aminotransferase >170 U/L, alanine-aminotransferase >140 U/L, cold ischemia time >12 hours, having had vasopressor drug such as dopamine dose >10 μg/kg/min or any does of other amines, and/or non-heart-beating; or (2) a donor after cardiac death with: an age >50 years, BMI>35 kg/m², a functional WIT>30 minutes, and/or macrosteatosis >30%. In other embodiments, an ECD liver donor refers to a deceased donor over the age of 60 with mild liver abnormalities; or upon recipient's informed consent to receive such an organ, a donor age 70 to 80, a donor older than age 60 with a significant medical history, a donor with a history of high-risk social behaviors, or a donor with a history of hepatitis B or C exposure.

An expanded criteria donor with respect to a pancreas donor commonly refers to a donor with an age of <10 or >45 (>50) years, BMI>30 kg/m², having had trauma in the pancreas, having had pancreatitis, having had alcohol intake, and/or donation after cardiac death. Generally standard pancreas donors are usually less than about 45 years of age and have a body mass index (BMI) of less than 30. A standard criteria donor pancreas come from a deceased donor who is declared brain dead. A donor pancreas can also come from a non-heart-beating donor, also known as donation after cardiac death (DCD). These are donors with severe brain injury with no hope for meaningful recovery and who do not meet the criteria for brain death. Another example of pancreas donor with extended criteria is from a donor with a BMI of 30 to 34 or a donor age between 50 and 60 years.

An expanded criteria donor with respect to a heart commonly refers to a donor with an age >40 (32) or >55 (33) years, BMI mismatch donor/recipient of >20%, hepatitis C virus positive, left ventricular hypertrophy >14 mm, ejection fraction <45%, having had high-dose catecholamine administration, having had tobacco or illicit drug use (cocaine), prolonged cardiopulmonary resuscitation, and/or transient reversible hypotension or cardiac arrest. Traditional cardiac donor criteria can be a donor with an age of less than 55 years old, no history of chest trauma or cardiac disease, no prolonged hypotension or hypoxemia, having appropriate hemodynamics (mean arterial pressure >60 mmHg and central venous pressure 8-12 mmHg), inotropic support of less than 10 mg/kg/min, normal electrocardiogram, normal echocardiogram, normal cardiac angiography, and a negative serology. An extended donor can expand in one or more aspects from the traditional criteria. Another example of extended cardiac donor criteria includes a donor with an age of >60 years, ECHO abnormalities, prolonged ischemic time, donor/recipient size mismatch >30%, positive blood/urine/sputum cultures, hepatitis B and/or C, significant pressor/inotrope requirements, donor substance abuse, long standing diabetes mellitus, cardiac artery disease, and/or structural cardiac abnormalities.

Donor inclusion criteria for intestinal procurement are described in Fischer-Frohlich et al., Transplant International, 25(12):1229-1240, December 2012, which is incorporated by reference herein.

Based on the standard criteria donor lung definitions published in 2003 by Orens et al., one can define an ECD lung if the donor does not fulfill at least one criterion of the standard criteria donor (SCD) criteria suggested by the International Society for Heart and Lung Transplantation. Common criteria defining standard criteria, therefore not ECD lungs: a donor with an age <55, BMI mismatch donor/recipient >20%, clear chest X-ray, PaO₂>300 mmHg (FIO2 1.0, PEEP 5 mm Hg), a history of smoking <20 pack years if any, absence of chest trauma, absence of microbiologic organisms endobronchial, absence of malignancy, absence of purulent secretions or signs of aspiration endobronchial, and negative virology. Hence, examples of ECD lung include those if the donor does not fulfill at least one criterion of the SCD criteria.

Traditional bone marrow donation inclusion criteria include a donor between the ages of 18 and 44, with no HIV (AIDS), no or little allergies to animals, the environment or medications, no or little osteoarthritis or degenerative arthritis, no asthma, no autoimmune illness that affects the whole body, no serious bleeding problem, no or manageable hypertension that is not associated with heart disease, no history of a significant brain injury or surgery in the brain tissue, no or some pre-cancerous cells that do not require chemotherapy, no or treated chemical dependency that has no physical ailments, no on-going, chronic, significant pain areas of the neck, back, hip, or spine, no heart disease, no serious or chronic kidney disease, no serious liver disease, and with a BMI that would not present a risk to the donor's safety. An expanded criteria bone marrow donor can be one that does not fulfill at least one criterion of the traditional inclusion criteria. Resch T, et al. Front Immunol. 2020; 11:631, Kukreja J, et al. Curr Opin Organ Transplant. 2020; 25(3):280-284, Pagano D, et al. Transplant Proc. 2020; 52(5):1588-1592, Noble J, et al. Front Immunol. 2020; 10:3142, Okamoto T, et al. Ann Thorac Surg. 2020; 109(6):1663-1669, Sommer W, et al. J Heart Lung Transplant. 2019; 38(5):560-569, and Schumer E M, et al. Ann Thorac Surg. 2015; 100(2):522-527, provide further description of ECD or marginal organs, which are incorporated by reference herein in the entireties.

C1 esterase inhibitor was approved by the United States Food and Drug Administration in 2009 for the treatment of hereditary angioedema. C1 esterase inhibitor is a serine protease inhibitor targeting C1s and C1r in the classical pathway and MASP-1 and MASP-2 in the MBL complement pathway and is therefore a relevant intervention to test the impact of complement inhibition on short- and long-term kidney function among allografts with ischemia-reperfusion injury. Described herein, we report a follow-up study of outcomes up to three and a half years from the original randomized-controlled trial of C1 esterase inhibitor versus placebo among patients at high risk for DGF.

In the study of a phase I/II randomized controlled trial of C1 esterase inhibitor versus placebo for the prevention of delayed graft function described herein, we found that treatment with C1 esterase inhibitor was associated with better graft survival and allograft function at three and a half years compared to placebo—including fewer graft losses developed and higher eGFR in the C1 esterase inhibitor group compared with placebo. Although the primary endpoint of DGF, defined as the need for dialysis within the first post-transplant week, was comparable between the two groups, the duration of dialysis-dependence was shorter in the C1 esterase inhibitor group. Follow-up data from the current study indicates that amelioration of ischemia-reperfusion injury with C1 esterase inhibitor leads to better long-term allograft outcomes that are significantly different from placebo controls in the study.

In the one-year follow-up report of this randomized controlled trial, there was no difference in graft survival at one year between C1 esterase inhibitor and placebo-treated patients. However, with longer-term follow-up, a difference in graft failure between the two groups was observed beginning after year one. At three and a half years, the cumulative incidence of graft failure was 21% among placebo-treated recipients, which is similar to graft failure statistics for KDPI >85% donor allografts reported nationally (16). This indicates that outcomes of placebo-treated recipients in this study were as expected for recipients of similar types of donors. In contrast, only one case of graft failure was observed among C1 esterase inhibitor-treated recipients despite no differences in baseline donor characteristics or chronicity scores on pre-implantation biopsies (p<0.03).

Furthermore, eGFR was consistently higher and sustained throughout the study period among C1 esterase inhibitor-treated recipients. This observation appeared to be independent of allograft rejection, as there were no differences in the number of rejection episodes or rejection types between the two groups. Altogether, these findings indicate that the classical and mannose-binding lectin complement pathways are important mediators of chronic allograft injury.

With C1 esterase inhibitor being protective against the long-term effects of ischemia-reperfusion injury in humans, there are important implications for deceased donor organ utilization. In the context of a critical donor organ shortage, approximately 20% of deceased donor kidneys that are recovered in the United States are not transplanted and discard rates exceed 50% for KDPI >85% allografts. Although the reasons for discard may vary, donor declines are driven in large part by concern over long-term viability of the allograft. With C1 esterase inhibitor leading to improved allograft function and long-term survival, its use could result in increased utilization of higher-risk donors and provide access to deceased donor transplantation for a significant number of waitlisted patients who would not otherwise receive a kidney transplant. As we demonstrate herein that C1 esterase inhibitor can lead to improved allograft function and long-term survival, its use could potentially result in increased utilization of higher-risk donors and provide access to deceased donor transplantation for a significant number of waitlisted patients who would not otherwise receive a kidney transplant.

The strength of our study was the randomized, double-blind, placebo-controlled trial design. The groups were well-matched on recipient and donor characteristics, and there were no differences in baseline graft histology. Therefore, it is less likely that the positive effects of C1 esterase inhibitor on later-term allograft function observed in this study were influenced by confounding factors.

In summary, administration of C1 esterase inhibitor to the recipients immediately prior to reperfusion and repeated 24 hours after transplant to recipients of deceased donor kidney allografts at high risk for DGF was associated with a shorter duration of dialysis-dependence, higher graft survival, and better allograft function over three and a half years compared to placebo-treated recipients. These findings indicate that inhibition of the classical and MBL complement pathways in kidneys susceptible to ischemia-reperfusion injury can mitigate allograft injury and lead to improved long-term allograft survival. Further, treatment with C1 esterase inhibitor can allow for broader utilization of higher-risk allografts.

Various embodiments of the present invention are based, in part, on these findings.

Various embodiments of the present invention provide for a method for improving long-term allograft survival in a subject in need thereof, comprising: administering to the subject a therapeutically effective amount of a C1 esterase inhibitor, wherein the subject will undergo allograft transplantation or has undergone kidney transplantation, and wherein the long-term allograft survival is more than 1 year after transplantation. In various embodiments, the method for improving the long-term allograft survival results in no graft failure, no detectable de novo donor specific antibodies, and/or improved glomerular filtration rate in 1 year, 2 years, 3 years, 3.5 years, 4 years, 4.5 years, 5 years, or longer after the graft transplantation to the subject.

In some embodiments, the allograft is a kidney, heart, liver, lung, small bowel, pancreas or bone marrow. In various embodiments, the allograft is a kidney.

In various embodiments, the C1 esterase inhibitor is complement C1 esterase inhibitor. In various embodiments, the C1 esterase inhibitor is plasma-derived BERINERT®, plasma-derived CINRYZE®, recombinant RUCONEST® or RHUCIN®.

In various embodiments, the therapeutically effective amount of C1 esterase inhibitor is administered on the day of the transplantation prior to reperfusion of the allograft.

In various embodiments, the therapeutically effective amount of C1 esterase inhibitor is administered about 24 hours after transplantation.

In various embodiments, administering to the subject comprises administering a first dose of a therapeutically effective amount of a C1 esterase inhibitor to the subject prior to reperfusion of the allograft and administering a second dose of a therapeutically effective amount of a C1 esterase inhibitor after transplantation of the allograft. In various embodiments, the first dose is administered to the subject intraoperatively prior to reperfusion of the allograft. In various embodiments, the second dose is administered about 24 hours after transplantation of the allograft.

In various embodiments, the effective amounts of one or more C1 esterase inhibitors are further administered to the subject one month, two months, six months, twelve months, 18 months, 24 months, 30 months, 36 months, 42 months, 48 months, or longer after transplant.

Typical dosages of an effective amount of one or more C1 esterase inhibitors can be in the ranges recommended by the manufacturer where known therapeutic compounds are used, and also as indicated to the skilled artisan by the in vitro responses or responses in animal models. For example, complement C1 esterase inhibitor (BERINERT®) is currently recommended at 50 Units/kg (rounded to the nearest 500 Units) intravenously. The same or similar dosing can be used in accordance with various embodiments of the present invention, or an alternate dosage may be used in connection with alternate embodiments of the invention. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments of the invention, the therapeutically effective amounts of one or more C1 esterase inhibitors for use with the methods described herein may be in the range of 1-5 units/kg, 5-10 units/kg, 10-20 units/kg, 20-30 units/kg, 30-40 units/kg, 40-50 units/kg, 10-50 units/kg, 50-60 units/kg, 60-70 units/kg, 70-80 units/kg, 80-90 units/kg, 90-100 units/kg, 50-100 units/kg, 100-150 units/kg, 150-200 units/kg, 100-200 units/kg, 200-300 units/kg, 300-400 units/kg, or 400-500 units/kg. In some embodiments, the therapeutically effective amount of C1 esterase inhibitor is about 25-50 units/kg, about 50-75 units/kg, about 75-100 units/kg or about 50 units/kg. In various embodiments, the therapeutically effective amount is about 50 units/kg. In various embodiments, the therapeutically effective amount is about 25-100 units/kg.

In some embodiments of the invention, the therapeutically effective amounts of one or more C1 esterase inhibitors can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In some embodiments of the invention, the therapeutically effective amounts of one or more C1 esterase inhibitors can be in the range of about 10-50 mg/period, 50-100 mg/period, 100-150 mg/period, 150-200 mg/period, 100-200 mg/period, 200-300 mg/period, 300-400 mg/period, 400-500 mg/period, 500-600 mg/period, 600-700 mg/period, 700-800 mg/period, 800-900 mg/period, 900-1000 mg/period, 1000-1100 mg/period, 1100-1200 mg/period, 1200-1300 mg/period, 1300-1400 mg/period, 1400-1500 mg/period, 1500-1600 mg/period, 1600-1700 mg/period, 1700-1800 mg/period, 1800-1900 mg/period, 1900-2000 mg/period, 2000-2100 mg/period, 2100-2200 mg/period, 2200-2300 mg/period, 2300-2400 mg/period, 2400-2500 mg/period, 2500-2600 mg/period, 2600-2700 mg/period, 2700-2800 mg/period, 2800-2900 mg/period or 2900-3000 mg/period.

In some embodiments, a period of time over which a dosing is administered is a day, a 36-hour timeframe, a 48-hour timeframe, a 72-hour timeframe, a week, two weeks, three weeks, four weeks, one month, two months, or three months. In some embodiments, the treated subject is administered the inhibitor over a time period of at least a year, two years, three years, or longer. In some embodiments, the C1 esterase inhibitor is administered for 2, 3, 4, 5, 6, 7, 8, 9, or 10 times over periods of one same length of time or different lengths of time in each period. In further embodiments, the C1 esterase inhibitor is administered in phases, wherein a break of not administering C1 esterase inhibitor exists between two consecutive phases of administration.

In various embodiments, the allograft is a kidney, the C1 esterase inhibitor is plasma-derived BERINERT®, the therapeutically effective amount is about 50 units/kg, the first dose is administered intraoperatively to the subject prior to reperfusion of the allograft, and the second dose is administered to the subject about 24 hours after transplantation of the allograft.

In various embodiments, the C1 esterase inhibitor is plasma-derived BERINERT®, the therapeutically effective amount is about 25, 30, 40, 50, 60, 70, 75, 80, 90 or 100 units/kg. In various embodiments, the therapeutically effective amount is about 50 units/kg. In various embodiments, the therapeutically effective amount is about 10-20, 20-30, 30-40, 40-50, 50-60, 60-70, 70-80, 80-90, or 90-100 units/kg of the subject.

In various embodiments, the C1 esterase inhibitor is administered intravenously or subcutaneously.

In various embodiments, the long-term allograft survival is at least 2, 2.5, 3, 3.5, 4, 4.5, 5, 5.5, 6, 6.5, 7, 7.5, 8, 8.5, 9, 9.5 or 10 years after transplantation.

In some embodiments, the treated subject is administered the C1 esterase inhibitor over a time period of at least a year. In some embodiments, the treated subject is administered the inhibitor over a time period of at least two years. In some embodiments, the treated subject is administered the inhibitor over a time period of at least three years. In some embodiments, the treated subject is administered the inhibitor over a time period of at least 3.5 years. Yet in some embodiments, the C1 esterase inhibitor is no longer administered after about three days, one week, or one month after the transplant, and the allograft function is improved at least for 3.5 years after the transplant.

In some embodiments, the subject exhibits signs of graft dysfunction and/or failure after graft transplant which is effectively treated, inhibited and/or reduced during the time period that the inhibitor is administered to the subject or after the inhibitor is administered to the subject.

In further embodiments, the C1 esterase inhibitor is administered both at the time of organ transplant (including prior to reperfusion and within 3 days of transplant) and after signs of delayed allograft function and/or graft dysfunction.

For a renal allograft, signs of graft dysfunction and/or failure include deterioration of allograft function as measured by an increase in serum creatinine level and/or a decrease in glomerular filtration rate; presence of donor-specific antibodies; biopsy evidence of capillaritis, inflammation and complement (C4d) deposition; and a need for dialysis.

In some embodiments, methods for improving long-term allograft survival, and/or reducing the severity or likelihood of, or providing prophylaxis against, allograft dysfunction, in a subject in need thereof, comprise administering to the subject a therapeutically effective amount of a C1 esterase inhibitor, wherein the subject is administered said C1 esterase inhibitor both at the time of allograft transplant and when the subject exhibits signs of delayed graft function and/or graft dysfunction which is effectively treated, inhibited and/or reduced during and after the time period that the C1 esterase inhibitor is administered to the subject.

In further embodiments, the methods further include measuring the allograft function in the subject, and continuing to administer one or more C1 esterase inhibitors if the allograft function is abnormal or deteriorates, or discontinuing the administration of the C1 esterase inhibitors if the allograft function is normal or has not deteriorated.

Some embodiments provide the methods further include selecting a subject that is at risk for delayed graft function (DGF), and administering to the subject an effective amount of one or more C1 esterase inhibitor, wherein the subject is in need of, undergoing, or having undergone an allograft transplant, and wherein said C1 esterase inhibitor is effective for an improved long-term graft survival, such as no graft failure in 3.5 years, 4 years, 5 years or longer after the transplant.

For renal transplant, a subject is at risk of developing DGF if: 1) he/she is a recipient of an allograft from an expanded criteria donor (ECD) or donor with a kidney donor profile index (KDPI) ≥85%; 2) recipient of an allograft from a donor classified as “donor after cardiac death” (DCD); or 3) recipient of an allograft with a risk index of three to eight for DGF based on traditional risk factors for DGF, such as those in Table 1. In some embodiments, the kidney allograft is not from a donor with brain death.

Various embodiments provide that for renal transplant recipients, the disclosed methods for improving long-term graft survival refers improvement to 1) no graft failure over 3.5 years after transplant; 2) no or little (less than 10% of recipients) de novo donor specific antibodies in at least 3.5 years after transplant; and/or 3) normal eGFR or improved eGFR compared to a control subject. A control subject may be one in need of renal transplant or having received a renal transplant but without receiving the C1 esterase inhibitor.

Various embodiments of the present invention provide for a method for improving long-term allograft function in a subject in need thereof, comprising: administering to the subject a therapeutically effective amount of a C1 esterase inhibitor, wherein the subject will undergo allograft transplantation or has undergone kidney transplantation, and wherein the long-term allograft function is more than 90 days after transplantation.

In some embodiments, the allograft is a kidney, heart, liver, lung, small bowel, pancreas or bone marrow. In various embodiments, the allograft is a kidney.

In various embodiments, the C1 esterase inhibitor is complement C1 esterase inhibitor. In various embodiments, the C1 esterase inhibitor is plasma-derived BERINERT®, plasma-derived CINRYZE®, recombinant RUCONEST® or RHUCIN®.

In various embodiments, the therapeutically effective amount of C1 esterase inhibitor is administered on the day of the transplantation prior to reperfusion of the allograft.

In various embodiments, the therapeutically effective amount of C1 esterase inhibitor is administered about 24 hours after transplantation.

In various embodiments, administering to the subject comprises administering a first dose of a therapeutically effective amount of a C1 esterase inhibitor to the subject prior to reperfusion of the allograft and administering a second dose of a therapeutically effective amount of a C1 esterase inhibitor after transplantation of the allograft. In various embodiments, the first dose is administered to the subject intraoperatively prior to reperfusion of the allograft. In various embodiments, the second dose is administered about 24 hours after transplantation of the allograft.

In various embodiments, the effective amounts of one or more C1 esterase inhibitors are further administered to the subject one month, two months, six months, twelve months, 18 months, 24 months or 30 months after transplant.

Typical dosages of an effective amount of one or more C1 esterase inhibitors can be in the ranges recommended by the manufacturer where known therapeutic compounds are used, and also as indicated to the skilled artisan by the in vitro responses or responses in animal models. For example, complement C1 esterase inhibitor (BERINERT®) is currently recommended at 50 Units/kg (rounded to the nearest 500 Units) intravenously. The same or similar dosing can be used in accordance with various embodiments of the present invention, or an alternate dosage may be used in connection with alternate embodiments of the invention. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments of the invention, the therapeutically effective amounts of one or more C1 esterase inhibitors for use with the methods described herein may be in the range of 1-5 units/kg, 5-10 units/kg, 10-20 units/kg, 20-30 units/kg, 30-40 units/kg, 40-50 units/kg, 10-50 units/kg, 50-60 units/kg, 60-70 units/kg, 70-80 units/kg, 80-90 units/kg, 90-100 units/kg, 50-100 units/kg, 100-150 units/kg, 150-200 units/kg, 100-200 units/kg, 200-300 units/kg, 300-400 units/kg, or 400-500 units/kg. In some embodiments, the therapeutically effective amount of C1 esterase inhibitor is about 25-50 units/kg, about 50-75 units/kg, about 75-100 units/kg or about 50 units/kg. In various embodiments, the therapeutically effective amount is about 50 units/kg. In various embodiments, the therapeutically effective amount is about 25-100 units/kg.

In some embodiments of the invention, the therapeutically effective amounts of one or more C1 esterase inhibitors can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day. In some embodiments of the invention, the therapeutically effective amounts of one or more C1 esterase inhibitors can be in the range of about 10-50 mg/period, 50-100 mg/period, 100-150 mg/period, 150-200 mg/period, 100-200 mg/period, 200-300 mg/period, 300-400 mg/period, 400-500 mg/period, 500-600 mg/period, 600-700 mg/period, 700-800 mg/period, 800-900 mg/period, 900-1000 mg/period, 1000-1100 mg/period, 1100-1200 mg/period, 1200-1300 mg/period, 1300-1400 mg/period, 1400-1500 mg/period, 1500-1600 mg/period, 1600-1700 mg/period, 1700-1800 mg/period, 1800-1900 mg/period, 1900-2000 mg/period, 2000-2100 mg/period, 2100-2200 mg/period, 2200-2300 mg/period, 2300-2400 mg/period, 2400-2500 mg/period, 2500-2600 mg/period, 2600-2700 mg/period, 2700-2800 mg/period, 2800-2900 mg/period or 2900-3000 mg/period. In some embodiments, a period of time over which a dosing is administered is a day, a 36-hour timeframe, a 48-hour timeframe, a 72-hour timeframe, a week, two weeks, three weeks, four weeks, one month, two months, or three months. In some embodiments, the treated subject is administered the inhibitor over a time period of at least a year, two years, three years, or longer. In some embodiments, the C1 esterase inhibitor is administered for 2, 3, 4, 5, 6, 7, 8, 9, or 10 times over periods of one same length of time or different lengths of time in each period. In further embodiments, the C1 esterase inhibitor is administered in phases, wherein a break of not administering C1 esterase inhibitor exists between two consecutive phases of administration.

In various embodiments, the allograft is a kidney, the C1 esterase inhibitor is plasma-derived BERINERT®, the therapeutically effective amount is about 50 units/kg, the first dose is administered intraoperatively to the subject prior to reperfusion of the allograft, and the second dose is administered to the subject about 24 hours after transplantation of the allograft.

In various embodiments, the C1 esterase inhibitor is plasma-derived BERINERT®, the therapeutically effective amount is about 25, 30, 40, 50, 60, 70, 75, 80, 90 or 100 units/kg. In various embodiments, the therapeutically effective amount is about 50 units/kg. In various embodiments, the therapeutically effective amount is in a range of 10-20, 20-30, 30-40, 40-50, 50-60, 60-70, 70-80, 80-90, or 90-100 units/kg of the subject, or in a range between any two values between 10 and 100 units/kg.

In various embodiments, the C1 esterase inhibitor is administered intravenously or subcutaneously.

In various embodiments, the long-term allograft function is at least 1, 1.5, 2, 2.5, 3, 3.5, 4, 4.5, 5, 5.5, 6, 6.5, 7, 7.5, 8, 8.5, 9, 9.5 or 10 years after transplantation.

In various embodiments, improved long-term allograft function includes increased estimated glomerular filtration rates, decreased serum creatinine levels, increased creatinine clearance, and/or increased urine output.

In some embodiments, the allograft (e.g., kidney) is from expanded criteria deceased donors, e.g., a donor over the age of 60, a donor over the age of 50 with two of the following: a history of high blood pressure, a creatinine (blood test that shows kidney function) greater than or equal to 1.5, or death resulting from a stroke.

In various embodiments, the therapeutically effective amounts of one or more C1 esterase inhibitors for use with the methods described herein may be administered at any one or more of the dosages described herein at least once 1-7 times per week, 1-7 times per month, 5-10 times per month or combinations thereof for 1 month, 2 months, 3 months, 4 months, 5 months 6 months, 7 months, 8 months, 9 months, 10 months, 11 months, 12 months, 14 months, 16 months, 18 months, 20 months, 22 months, 24 months or combinations thereof.

In further embodiments of the methods, a pharmaceutical composition including a C1 esterase inhibitor and a pharmaceutically acceptable excipient is administered in an effective amount to a subject in need thereof to improve the long-term allograft survival. In some embodiments, the subject is administered said C1 esterase inhibitor over a time period of more than 2 years (e.g., at least 2 years, 2.5 years, 3 years, 3.5 years, 4 years, 5 years, or longer) and the subject exhibits signs of graft dysfunction and/or failure after graft transplant which is effectively treated, inhibited and/or reduced during and/or after the time period that the C1 esterase inhibitor is administered to the subject. In further embodiments, the C1 esterase inhibitor is no longer administered after about 3 days, 4 days, 5 days, 6 days, 1 week, or 1 month after the transplant, and the allograft function is improved for at least 1 year, 2 years, 3 years, or 3.5 years.

“Pharmaceutically acceptable excipient” includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In further embodiments, the pharmaceutical compositions contain a C1 esterase inhibitor and a pharmaceutically acceptable carrier, which may be a liquid or solid filler, diluent, excipient, solvent, or encapsulating material, or a combination thereof. Before administration to the subject, formulants may be added to the composition comprising the C1 esterase inhibitor; and exemplary formulants include oils, polymers, vitamins, carbohydrates, amino acids, salts, buffers, albumin, surfactants, bulking agents or combinations thereof.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The clinical and research activities being reported are consistent with the Principles of the Declaration of Istanbul as outlined in the “Declaration of Istanbul on Organ Trafficking and Transplant Tourism”. This study was approved by the Cedars-Sinai Institutional Review Board and the conduct of the study adhered to the principles of the Declaration of Helsinki. Briefly, the original study, performed at Cedars-Sinai Medical Center in Los Angeles, Calif., was a phase I/II double-blind, randomized, placebo-controlled trial investigating the safety and efficacy of C1 esterase inhibitor for prevention of DGF after deceased-donor kidney transplantation (NCT02134314; date of registration May 9, 2014). All enrolled subjects provided written consent for participation in the trial. Eligibility criteria included patients aged 18-70 years old with end-stage kidney disease (ESKD) requiring maintenance dialysis awaiting kidney transplantation. Patients were considered to be at high-risk for DGF if they met the following eligibility criteria: 1) recipient of an allograft from an expanded criteria donor (ECD) or donor with a kidney donor profile index (KDPI) >85%; 2) recipient of an allograft from a donor classified as “donor after cardiac death” (DCD); or 3) recipient of an allograft with a risk index of three to eight for DGF (Table 1). This index was based on traditional risk factors for DGF and a published nomogram by Irish, et al., J. Am. Soc. Nephrol., 14:2967-2974, 2003.

TABLE 1
Risk Factors for Delayed Graft Function

| Characteristic | Risk Index* |
|---|---|
| Donor Age (years) | |
| <40 | 0 |
| 41-49 | 1 |
| 50-54 | 2 |
| 55-59 | 3 |
| ≥60 | 6 |
| Cold Ischemia Time (hours) | |
| 0-12 | 0 |
| 13-18 | 1 |
| 19-24 | 2 |
| 24-30 | 3 |
| 31-36 | 4 |
| ≥37 | 6 |
| Recipient Race | |
| Non-Black | 0 |
| Black | 1 |
| Recipient with Diabetes | |
| Yes | 1 |
| No | 0 |
| Donor Cause of Death Due to cerebrovascular accident | |
| Donor age < 50 years | 0 |
| Donor age ≥ 50 years | 3 |
| Donor Terminal Creatinine | |
| ≥4 mg/dL | 3 |
| ≥2-4 mg/dL | 2 |
| <2 mg/dL | 0 |

*Patients with a total risk index score of 3-8 qualified for the trial; expanded criteria and donor after cardiac death donors automatically qualified for the trial.

### Study Design and Treatment

From Nov. 25, 2014 through Dec. 29, 2016, a total of 70 patients with ESKD on dialysis were randomized 1:1 to receive C1 esterase inhibitor 50 U/kg (N=35) or placebo (normal saline; N=35) given in the operating room prior to reperfusion of the kidney allograft and repeated 24 hours later. The study treatment was prepared according to a pharmacist-supplied randomization code that was blinded from all other study personnel. Per study protocol, patients underwent a preimplantation allograft biopsy before administration of study medication.

Antibody induction consisted of alemtuzumab 30 mg by subcutaneous injection post-operatively (for highly-sensitized recipients, defined as having calculated panel reactive antibodies >30% or recipient of a prior transplant) or anti-thymocyte globulin 1.5 mg/kg daily for four doses. Maintenance immunosuppression consisted of tacrolimus or cyclosporine, mycophenolate mofetil, and corticosteroids per center protocol. In addition to standard antimicrobial prophylaxis against Pneumocystis jirovecii, cytomegalovirus, and fungal infections given according to local institutional guidelines, all patients were given meningococcal vaccination before transplantation and received antibiotic prophylaxis directed against Neisseria meningitides for one month post-transplant.

The primary outcome of the original study was the proportion of patients with DGF, defined as the need for dialysis in the first week after transplant, excluding dialysis in the first 24 hours for hyperkalemia or volume overload. Exploratory outcomes of the original study included patient and graft survival at twelve months and eGFR at twelve months.

### Statistical Analysis

The present study was a post-hoc analysis investigating outcomes up to three and a half years among the original cohort of trial participants from the randomized trial. Exploratory outcomes were death-censored graft failure, patient survival, and change in eGFR over time. The cumulative incidence functions method was used to assess time to graft failure and death using the Gray test for statistical comparison. The Kaplan-Meier product-limit method was used to assess for time to patient survival, with statistical comparisons made using the log-rank test. In order to account for the competing risk of death, time to graft failure, defined as return to dialysis, was assessed using the cumulative incidence functions method and using the Gray's test for statistical comparison.

Graft failure was defined as return to dialysis, and the date of return to dialysis was considered as the date of graft failure. Patients were censored at the end of the study period, with last follow-up date on Nov. 21, 2018. For comparison of eGFR, a linear mixed effects model was employed using random slopes and intercepts and an unstructured covariance matrix to compare the slope difference in eGFR between C1 esterase inhibitor and placebo-treated recipients. eGFR was estimated using the Chronic Kidney Disease Epidemiology Collaboration creatinine equation. The fixed effect represents the average change in eGFR by treatment group, whereas the random effect accounts for subject-specific correlation between repeated measures of eGFR within an individual. The intent of this analysis was to compare long-term changes in eGFR between the two groups. Given that early allograft dysfunction in the setting of DGF could erroneously impact the modeled slope, only eGFR values obtained more than one month post-transplant were used for the analysis. All eGFR values up to the date of graft failure or death were included in the model and no eGFR values were imputed.

P-values were two-tailed and a p<0.05 was considered statistically significant. All analyses were performed using Stata version 14.2 (College Station, Tex.) and R version 3.5.1.

### Results

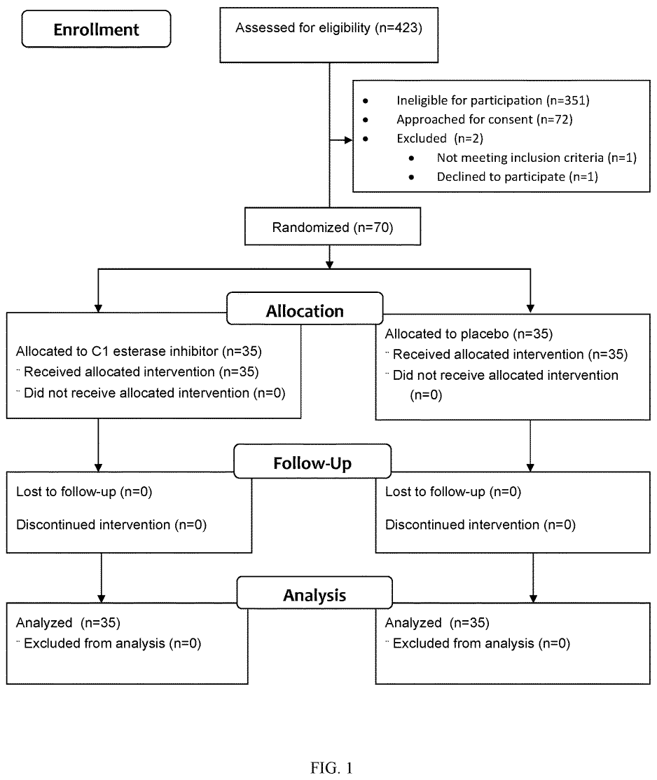

Between Nov. 25, 2014 and Dec. 29, 2016, a total of 70 deceased donor kidney transplant recipients were enrolled in the study, with 35 recipients randomized to receive C1 esterase inhibitor and 35 randomized to receive placebo (FIG. 1). The median follow-up time of participants was 2.5 years (interquartile range [IQR]: 2.0-3.0 years). Baseline characteristics are shown in Table 2. The groups were well-matched with regard to recipient, donor, and immunologic characteristics. Specifically, the number of donor after cardiac death, mean KDPI, and the number of donors with KDPI >85% were similar among C1 esterase inhibitor and placebo-treated recipients. Additionally, the groups were similar in the degree of HLA allosensitization and donor-recipient cross-matching. A comparison of baseline pre-implantation allograft biopsies was not different between C1 esterase inhibitor and placebo-treated recipients in histologic scoring for glomerulosclerosis, interstitial fibrosis/tubular atrophy, arteriosclerosis, and arteriolar hyalinosis. There was no difference in the original trial's primary outcome of DGF between the two groups, defined as the need for dialysis in the first post-transplant week (C1 esterase inhibitor 44% vs. placebo 60%, p=0.23), nor was there a difference in the mean number of dialysis sessions required (C1 esterase inhibitor 1+/−1 vs. placebo 2+/−2, P=0.12). However, exploratory analyses revealed that the duration of DGF was shorter in C1 esterase inhibitor-treated patients and all patients in the C1 esterase inhibitor group discontinued dialysis by two weeks post-transplant, whereas 5 (14%) patients in the placebo arm remained dialysis-dependent beyond two weeks (p=0.02) (13).

TABLE 2
Baseline characteristics.

| | | | C1 esterase | | | |
|---|---|---|---|---|---|---|
| | All Patients | | inhibitor | | Placebo | |
| | (n = 70) | | (n = 35) | | (n = 35) | |
| Recipient Characteristics | | | | | | |
| Age, years, mean (SD) | 58 | (10) | 58 | (8) | 58 | (11) |
| Male (%) | 42 | (69) | 20 | (57) | 22 | (63) |
| Diabetes mellitus (%) | 41 | (59) | 21 | (60) | 20 | (57) |
| Black (%) | 8 | (11) | 4 | (11) | 4 | (11) |
| Donor Characteristics | | | | | | |
| Age, years, mean (SD) | 47 | (14) | 49 | (12) | 46 | (16) |
| Creatinine, mg/dL, mean (SD) | 1.8 | (1.7) | 1.8 | (1.4) | 1.8 | (1.9) |
| Donor after Cardiac Death (%) | 23 | (33) | 11 | (31) | 12 | (34) |
| KDPI, mean (SD) | 66 | (24) | 68 | (22) | 65 | (25) |
| KDPI ≥ 85% (%) | 21 | (30) | 10 | (29) | 11 | (31) |
| Transplant Characteristics | | | | | | |
| Cold ischemia time, hours, mean (SD) | 19 | (6) | 18 | (5) | 20 | (6) |
| Immunologic Time Characteristics | | | | | | |
| Presence of anti-HLA DSA (%) | 5 | (7) | 3 | (9) | 2 | (6) |
| Immunodominant Class I | 3 | (4) | 2 | (6) | 1 | (3) |
| Immunodominant Class II | 2 | (3) | 1 | (3) | 1 | (3) |
| DSA MFI, mean (SD) | 5750 | (1425) | 5000 | (1250) | 6875 | (884) |
| Positive crossmatch (%) | 2 | (3) | 1 | (3) | 1 | (3) |
| Antibody Induction | | | | | | |
| Anti-thymocyte globulin (%) | 53 | (76) | 25 | (71) | 28 | (80) |
| Alemtuzumab (%) | 17 | (24) | 10 | (29) | 7 | (20) |

Abbreviations: KDPI: kidney donor profile index; DSA: donor-specific antibodies; MFI: mean fluorescence intensity.

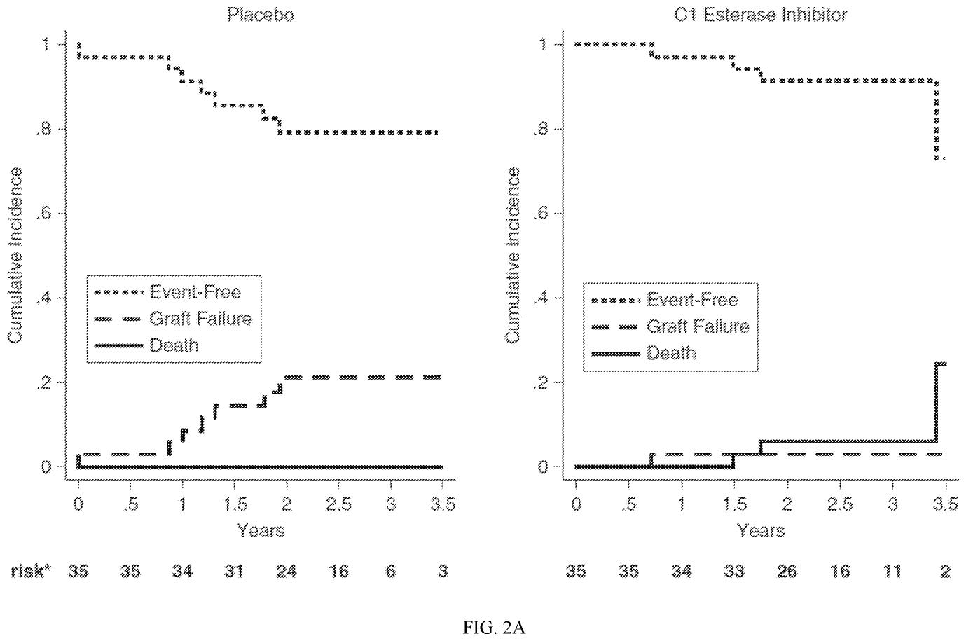

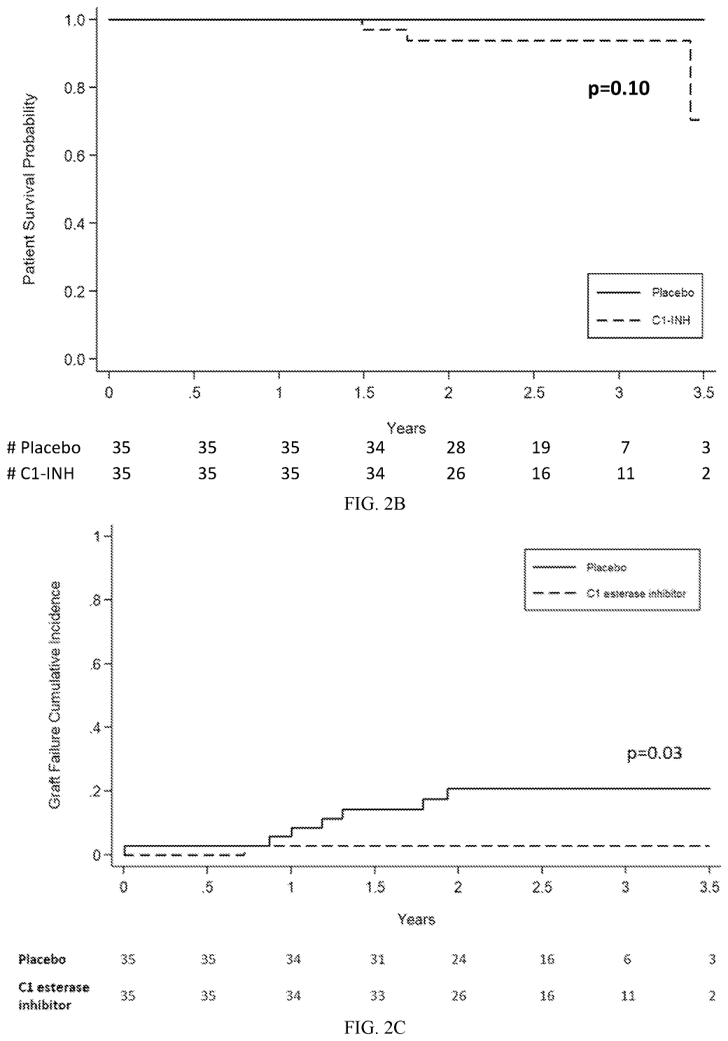

FIG. 2A shows the cumulative incidence of death and graft failure with corresponding event-free survival in the placebo and C1 esterase inhibitor groups. The cumulative incidence of death was not statistically different between the two groups over the study period (P value by Gray test=0.09). There were three deaths, all within the C1 esterase inhibitor group, each occurring >1.5 years after transplant. Given the time interval post-transplant, these deaths were considered to be unrelated to C1 esterase inhibitor. Death was due to a bacterial infection in two patients, one of whom had received intensified immuno-suppression as treatment for antibody-mediated rejection. The third patient was also treated for antibody-mediated rejection and died due to an unknown cause. All three patients had a functioning allograft at the time of death.

There were seven graft failures observed in the placebo group compared with one in the C1 esterase inhibitor group, all occurring within the first 2 years post-transplant. The cumulative incidence of graft failure was significantly higher over 3.5 years among placebo-treated patients compared with C1 esterase inhibitor-treated recipients (P value by Gray test=0.03; unadjusted hazard ratio [HR], 0.14; 95% confidence interval [95% CI], 0.02 to 1.15). At 3.5 years, the cumulative incidence of graft failure was 21% among placebo-treated patients and 3% among C1 esterase inhibitor-treated patients.

When death and graft loss were considered together, there was no association between treatment group (C1 esterase inhibitor versus placebo) and event-free survival over the study period (unadjusted HR, 0.53; 95% CI, 0.16 to 1.82).

FIG. 2B compares patient survival up to three and a half years between the placebo and C1 esterase inhibitor groups. Patient survival was not statistically different between the two groups over the study period (log-rank p=0.10). There were three deaths, all within the C1 esterase inhibitor group, occurring more than one and a half years after transplant. Given the time interval post-transplant, these deaths were considered not to be related to C1 esterase inhibitor. Death was due to a bacterial infection in two patients, one of whom had received intensified immunosuppression as treatment for antibody-mediated rejection. The third patient was also treated for antibody-mediated rejection and died due to an unknown cause. All three patients had a functioning allograft at the time of death.

FIG. 2C compares the cumulative incidence of graft failure with follow-up to three and a half years. There were seven graft failures observed in the placebo group compared to one in the C1 esterase inhibitor group, all occurring within the first two years post-transplant. The cumulative incidence of graft failure was significantly higher over three and a half years among placebo-treated patients compared to C1 esterase inhibitor-treated recipients (p value by Gray's test=0.03; unadjusted sHR: 0.14, 95% CI: 0.02-1.15). At three and a half years, the cumulative incidence of graft failure was 21% among placebo-treated patients and 3% among C1 esterase inhibitor-treated patients.

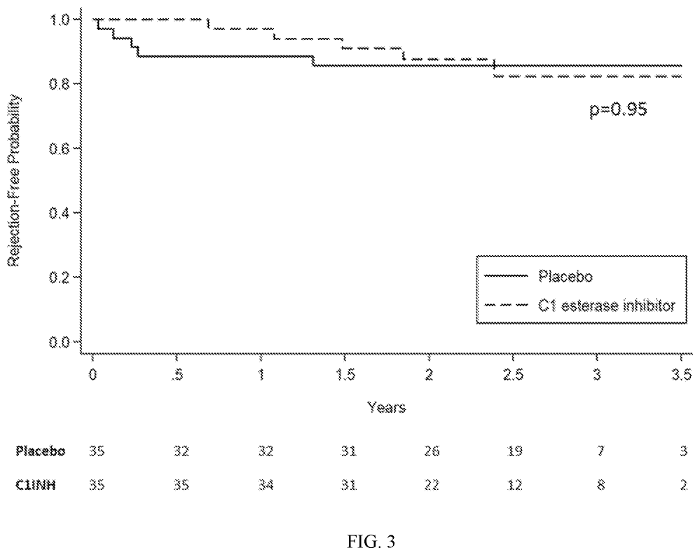

There was no difference in rejection-free survival between the C1 esterase inhibitor and placebo groups over three and a half years (FIG. 3; log-rank p=0.97; unadjusted HR: 0.96, 95% CI: 0.28-3.33). At three and a half years, rejection-free survival was 82% in the C1 esterase inhibitor group and 86% in the placebo group. Five patients with rejection were observed in each group. The distribution of rejection types was similar in both groups. Among C1 esterase inhibitor-treated recipients, there were two cases of isolated cell-mediated rejection in patients, two cases of chronic active antibody-mediated rejection that developed in recipients who were HLA-sensitized, and one case of mixed cell-mediated and chronic-active antibody-mediated rejection in a recipient of a prior organ transplant. Within the placebo group, there were two cases of isolated cell-mediated rejection in patients, one case of isolated antibody-mediated rejection that developed in a recipient of an HLA-incompatible kidney transplant, and two cases of mixed cell-mediated and antibody-mediated rejection. De novo donor specific antibodies (DSA) developed in 8 (23%) placebo-treated and 3 (9%) C1 esterase inhibitor-treated recipients (p=0.10). There was no difference in time to de novo DSA between the two groups [median months, placebo: 14 (IQR: 9-15); C1 esterase inhibitor: 19 (IQR: 2-27); p=0.68].

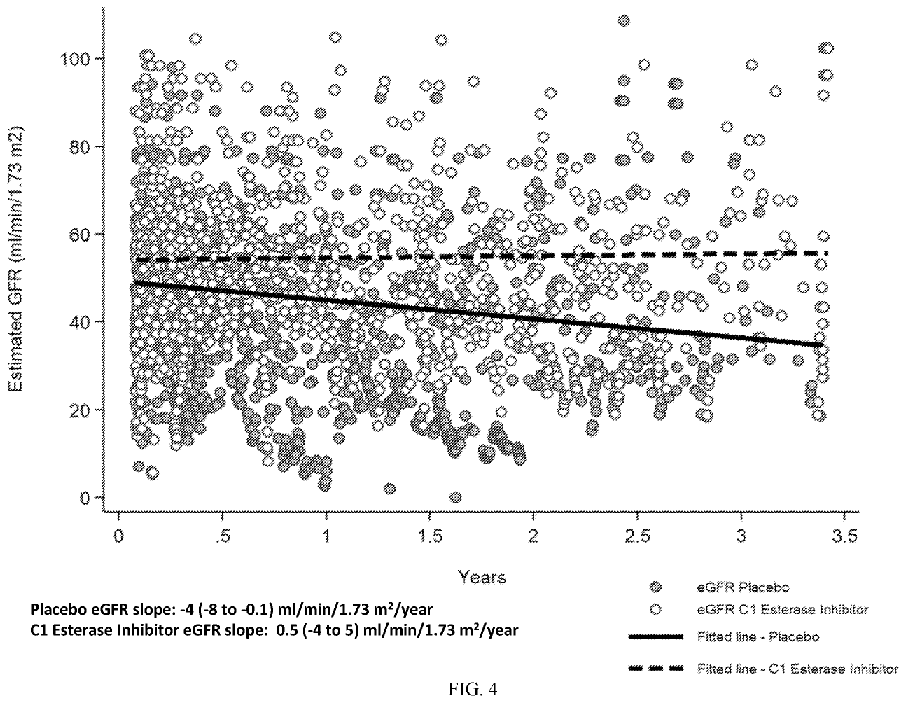

FIG. 4 compares the model-based predicted eGFR slope over time between C1 esterase inhibitor- and placebo-treated recipients. At 1 month post-transplant, the eGFR was 49 ml/min per 1.73 m² (95% CI, 44 to 54 ml/min per 1.73 m²) in the placebo group, which was not statistically different than the C1 esterase inhibitor group (54 ml/min per 1.73 m²; 95% CI, 49 to 59 ml/min per 1.73 m²; P=0.15). A significant difference in eGFR slope was not observed between the C1 esterase inhibitor and placebo groups [coefficient for treatment group x time interaction: 5 ml/min/1.73 m²/year (95% CI: −1 to 10 ml/min per 1.73 m² per year; P=0.12)]. However, within each treatment group, eGFR slopes were statistically meaningful; the trajectory of eGFR was different in each group. In the C1 esterase inhibitor group, the estimated mean eGFR did not change over the study period [eGFR slope=0.5 ml/min per 1.73 m² per year (95% CI: −4 to 5 ml/min per 1.73 m² per year)]. In contrast, the estimated mean eGFR decreased in the placebo group [eGFR slope=−4 ml/min per 1.73 m² per year (95% CI: −8 to −0.1 ml/min per 1.73 m² per year)]. At three and a half years, the estimated mean eGFR was higher among C1 esterase inhibitor-treated recipients (56 ml/min per 1.73 m² (95% CI: 42-70 ml/min per 1.73 m²) compared with placebo (35 ml/min per 1.73 m²; 95% CI, 21 to 48 ml/min per 1.73 m²), with a mean eGFR difference between the two groups of 21 ml/min per 1.73 m² (95% CI, 2 to 41 ml/min per 1.73 m²). The estimated mean eGFR was 49 ml/min/1.73 m² (95% CI: 45 to 54 ml/min/1.73 m²) at one month post-transplant and decreased to 35 ml/min/1.73 m² (95% CI: 21 to 48 ml/min/1.73 m²) at three and a half years post-transplant. Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method for improving long-term allograft survival, or improving long-term allograft function, in a subject in need thereof, comprising:
administering to the subject a therapeutically effective amount of a C1 esterase inhibitor,
wherein the subject will undergo allograft transplantation or has undergone kidney transplantation, and
wherein the long-term allograft survival is more than 1 year after transplantation, and wherein the long-term allograft function is more than 90 days after transplantation.

2. The method of claim 1, wherein the allograft is a kidney.

3. The method of claim 1, wherein the allograft is from an expanded criteria deceased donor.

4. (canceled)

5. The method of claim 1, wherein the C1 esterase inhibitor is plasma-derived BERINERT®, plasma-derived CINRYZE®, recombinant RUCONEST® or RHUCIN®.

6. The method of claim 1, wherein the therapeutically effective amount of C1 esterase inhibitor is administered on the day of the transplantation prior to reperfusion of the allograft.

7. The method of claim 1, wherein the therapeutically effective amount of C1 esterase inhibitor is administered about 24 hours after transplantation.

8. The method of claim 1, wherein administering to the subject comprises administering a first dose of a therapeutically effective amount of a C1 esterase inhibitor to the subject prior to reperfusion of the allograft and administering a second dose of a therapeutically effective amount of a C1 esterase inhibitor after transplantation of the allograft.

9. The method of claim 8, wherein the first dose is administered to the subject intraoperatively prior to reperfusion of the allograft, or wherein the second dose is administered about 24 hours after transplantation of the allograft.

10. (canceled)

11. The method of claim 1, wherein the therapeutically effective amount is about 25-100 units/kg.

12. The method of claim 1, wherein the therapeutically effective amount is about 50 units/kg.

13. The method of claim 8, wherein
the allograft is a kidney,
the C1 esterase inhibitor is plasma-derived BERINERT®,
the therapeutically effective amount is about 50 units/kg, and
the first dose is administered intraoperatively to the subject prior to reperfusion of the allograft, and
the second dose is administered to the subject about 24 hours after transplantation of the allograft.

14. The method of claim 1, wherein the C1 esterase inhibitor is administered intravenously or subcutaneously.

15. The method of claim 1, for improving the long-term allograft survival, wherein the long-term allograft survival is at least 2 years after transplantation.

16. The method of claim 1, for improving the long-term allograft survival, wherein the long-term allograft survival is at least 3.5 years after transplantation.

17-30. (canceled)

31. The method of claim 1, for improving the long-term allograft function, wherein the long-term allograft function is at least 1 years after transplantation.

32. The method of claim 1, for improving the long-term allograft function, wherein the long-term allograft survival is at least 3.5 years after transplantation.

33. A method for improving long-term allograft function, in a subject in need thereof, comprising:
administering to the subject a therapeutically effective amount of a C1 esterase inhibitor,
wherein the subject is at risk of developing delayed graft function (DGF), or the subject exhibits DGF or signs of graft dysfunction after allograft transplant which is effectively treated, inhibited and/or reduced after the C1 esterase inhibitor is administered to the subject, and
wherein the long-term allograft survival is more than 1 year after transplantation.

34. The method of claim 33, wherein after the allograft transplant, the subject exhibits one or more signs of graft dysfunction selected from the group consisting of: (i) deterioration of allograft function as measured by an increase in serum creatinine level and/or a decrease in glomerular filtration rate; (ii) presence of donor-specific antibodies; (iii) biopsy evidence of capillaritis, inflammation and complement (C4d) deposition; and (iv) a need for dialysis, or wherein the subject is at risk of developing delayed graft function, characterized by the subject being (i) a recipient of an allograft from an expanded criteria donor (ECD) or donor with a kidney donor profile index that is at least 85%; (ii) recipient of an allograft from a donor after cardiac death (DCD); or (iii) recipient of an allograft with a risk index of three to eight for delayed graft function.

35. (canceled)

36. The method of claim 33, wherein
the allograft is a kidney,
the C1 esterase inhibitor is plasma-derived BERINERT®,
the therapeutically effective amount is about 25-50 units/kg, and
a first dose of the C1 esterase inhibitor is administered intraoperatively to the subject prior to reperfusion of the allograft, and
a second dose of the C1 esterase inhibitor is administered to the subject about 24 hours after transplantation of the allograft.

37. The method of claim 36, wherein the C1 esterase inhibitor is not administered after the second dose, or the C1 esterase inhibitor is no longer administered after about three days after transplant, and the allograft function is improved at least for 3.5 years after the transplant, and wherein the improved allograft function at least for 3.5 years after the transplant includes no graft failure, no detectable de novo donor specific antibodies, and/or normal estimated glomerular filtration rates at 3.5 years after the transplant.

38-39. (canceled)
